(19) United States
(12) Patent Application Publication
(10) Pub. No.: US 2019/0292606 A1
(43) Pub. Date: Sep. 26, 2019

(54) IMMUNE CELL SIGNATURES

(71) Applicant: NantOmics, LLC, Culver City, CA (US)

(72) Inventors: Christopher W. SZETO, Culver City, CA (US); Sandeep K. REDDY, Culver City, CA (US)

(21) Appl. No.: 16/420,605

(22) Filed: May 23, 2019

Related U.S. Application Data
(63) Continuation-in-part of application No. 16/358,576, filed on Mar. 19, 2019.
(60) Provisional application No. 62/647,621, filed on Mar. 23, 2018.
(60) Provisional application No. 62/676,510, filed on May 25, 2018.

(51) Int. Cl. C12Q 1/6886; A61P 35/00; G16H 50/30; G16H 50/20

(52) U.S. Cl. CPC C12Q 1/6886; A61P 35/00; G16H 50/30; C12Q 2600/156; C12Q 2600/112; C12Q 2600/118; C12Q 2600/106; G16H 50/20

(57) ABSTRACT

An immune gene expression signature is associated with clinical features in tumor samples and can be used to predict the immunological state of a tumor and/or sensitivity of the tumor to immune therapy.

This application is a continuation in part of co-pending U.S. application Ser. No. 16/358,576 filed on Mar. 19, 2019, which claims benefit of priority to U.S. provisional applications with the Ser. No. 62/647,621, filed Mar. 23, 2018. This application also claims the benefit of priority to our co-pending U.S. provisional application 62/676,510 filed on May 25, 2018. Each of these applications are incorporated by reference in its entirety herein.

### FIELD OF THE INVENTION

The field of the invention is genetic analysis of tumor tissue, especially as it relates to immune cells signatures.

### BACKGROUND OF THE INVENTION

The background description includes information that may be useful in understanding the present invention. It is not an admission that any of the information provided herein is prior art or relevant to the presently claimed invention, or that any publication specifically or implicitly referenced is prior art.

All publications and patent applications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Studies of the tumor microenvironment have surfaced promising avenues of exploration to better understand the clinical relevance of T cell immune biology. Regulatory T cells (Tregs) have keenly emerged in light of their ability to inhibit the adaptive immune response and provide a mechanism of immune escape for cancer cells within the tumor microenvironment across various cancer types. However, the relatively large number of studies exploring the clinical relevance of intratumoral Treg abundance has produced controversial results to date, with some studies finding a poor prognosis associated with Treg infiltration, and others suggesting a favorable Treg-associated prognosis. Not surprisingly, the recent efforts to account for these polarized clinical results have undermined the notion that FOXP3+ Tregs invariably suppress tumor immunity. To address this uncertainty, multiple gene markers were taken into account to more accurately identify Tregs, such as FOXP3+BLIMP1 or FOXP3+CTLA4. However, none of the known studies have produced results that were suitable to guide a clinician towards a rational-based therapy with high confidence in a predicted outcome.

Indeed, immune heterogeneity within the tumor microenvironment has added multiple layers of complexity to the understanding of chemosensitivity and survival across various cancer types. Within the tumor microenvironment, immunogenicity is a favorable clinical feature in part driven by the antitumor activity of CD8+ T cells. However, tumors often inhibit this antitumor activity by exploiting the suppressive function of Regulatory T cells (Tregs), thus suppressing an adaptive immune response.

Unfortunately, there are numerous mechanisms other than Tregs and CD8+T involved in the immunogenicity of tumor cells, and an accurate prediction of immunogenicity of a tumor has remained elusive. Indeed, it has been reported that the immune infiltrate composition changes at each tumor stage and that particular immune cells have a major impact on survival. For example, densities of T follicular helper (Tfh) cells and innate cells increases, and most T cell densities decrease where tumor progression is observed. Moreover, the number of B cells, which are key players in the core immune network and are associated with prolonged survival, increase at a late stage and often show a dual effect on recurrence and tumor progression (see e.g., Immunity 2013 Oct. 17; 39(4):782-95).

Therefore, despite numerous findings in isolation, complex interactions between tumors and their microenvironment remain to be elucidated. Consequently, there is still a need for improved systems and methods to better characterize immunogenicity of a tumor.

### SUMMARY OF THE INVENTION

The inventive subject matter is directed to various methods of genetic analysis, and especially quantitative and normalized RNA expression analysis of tumor tissue, to thereby allow for identification of infiltration and/or activity of various immune cells in a specific tumor. For example, in some embodiments, the inventors used various gene sets associated with various immune cells types and then correlated them with specific disease categories (e.g., ICD10 categories) to predict whether or not a tumor is immune-enriched. Moreover, immune cell-enrichment was found to be correlated with PDL1 high/normal/low cases, and molecular targets could also be identified for patients where PDL1 is low.

In one aspect of the inventive subject matter, the inventors contemplate a method of characterizing a tumor that includes a step of quantifying or obtaining expression levels for a plurality of distinct genes, wherein the distinct genes are associated with (e.g., expressed in, most typically specifically expressed in) respective distinct types of immune cells, and a further step of determining over-expression or under-expression for each of the distinct genes relative to respective reference ranges, wherein the reference ranges are specific for a specific tumor type. In yet another step the over-expression and/or under-expression of each of the distinct genes is then used to infer activity and/or infiltration by the immune cells in the tumor.

Most typically, but not necessarily, the expression level is measured via qPCR or RNAseq, and suitable genes for such analysis include BLK, CD19, CR2 (CD21), HLA-DOB, MS4A 1 (CD20), TNFRSF17 (CD269), CD2, CD3E, CD3G, CD6, ANP32B (APRIL), BATF, NUP107, CD28, ICOS (CD278), CD38, CSF2 (GM-CSF), IFNG, IL12RB2, LTA, CTLA4 (CD152), TXB21, STAT4, CXCR6 (CD186), GATA3, IL26, LAIR2 (CD306), PMCH, SMAD2, STATE, IL 17A, IL 17RA (CD217), RORC, CXCL13, MAF, PDCD1 (CD279), BCL6, FOXP3, ATM, DOCKS, NEFL, REPS1, USP9Y, AKT3, CCR2 (CD192), EWSR1 (EWS), LTK, NFATC4, CD8A, CD8B, FLT3LG, GZMM, MET1, PRF1, CD160, FEZ1, TARP (TCRG), BCL2, FUT5, NCR1 (CD335), ZNF205, FOXJ1, MPPED1, PLA2G6, RRAD, GTF3C1, GZMB, IL21R (CD360), CCL13, CCL17, CCL22 (MDC), CD209, HSD11B1, CD1A, CD1B, CD1E, F13A1, SYT17, CCL1, EBI3, IDO1 (INDO), LAMP3 (CD208), OAS3, IL3RA (CD123), APOE, CCL 7 (FIC), CD68, CHIT1, CXCL5, MARCO, MSR1 (CD204), CMA1, CTSG, KIT (CD117), MS4A2, PRG2, TPSAB1, CSF3R (CD114), FPR2, MME (CD10), CCR3 (CD193), IL5RA (CD125), PTGDR2, (CD294), SMPD3, and THBS1.

In further contemplated embodiments, a threshold for determination of over-expression or under-expression may be when the quantified expression level exceeds +/−2SD of the reference range. Most preferably, the reference range is specific for a particular tumor type as classified in ICD10. As will be readily appreciated, the immune status may then be associated with the tumor based on the inferred activity and/or infiltration. Consequently, immune therapy such as treatment with a checkpoint inhibitor, treatment with immune stimulatory compositions, and/or vaccination with a tumor associated antigen or tumor and patient specific may then be recommended or initiated. For example, checkpoint inhibitor treatment with a PDL1 inhibitor may be used for a PDL1-high tumor, while checkpoint inhibitor treatment with a TIM3 inhibitor or an IDO inhibitor may be recommended or initiated for a PDL1-low tumor.

Therefore, viewed from a different perspective, the inventor also contemplates a method of identifying a patient for immune therapy that will include a step of quantifying or obtaining expression levels for a plurality of distinct genes, wherein the distinct genes are associated with respective distinct types of immune cells. In a further step, over-expression or under-expression is determined for each of the distinct genes relative to respective reference ranges, wherein the reference ranges are specific for a specific tumor type, and in yet another step, the over-expression and/or under-expression of each of the distinct genes is used to infer activity and/or infiltration by the immune cells in the tumor. The so inferred activity is then used to predict an increased likelihood of positive treatment outcome where the inferred activity and/or infiltration of distinct immune cells in the tumor is increased relative to the respective reference ranges, and the patient is selected or identified as a suitable candidate for immune therapy upon prediction of the increased likelihood.

For example, the distinct immune cells in the tumor include pDC, aDC, TFH, NK cells, neutrophils, Treg, iDC, macrophages, Thelper cells, NK cells, CD8 T cells, T cells, and Th1 cells, and/or the increased number may be with respect to at least three or four distinct types of immune cells in the tumor. Suitable genes for such analysis include those noted above, and over-expression or under-expression may be ascertained when the quantified expression level exceeds +/−2SD of the reference range. As will be readily appreciated, suitable immune therapies include treatment with a checkpoint inhibitor, a vaccine composition, and/or an immune stimulatory cytokine.

Therefore, the inventor also contemplates the use of a plurality of distinct genes to characterize a tumor or to predict treatment outcome for immune therapy of the tumor, wherein the plurality of distinct genes are associated with respective distinct types of immune cells, and wherein the use comprises a quantification of expression levels of the distinct genes. Once more, suitable genes for such analysis include those noted above, and over-expression or under-expression for each of the distinct genes is preferably determined relative to respective reference ranges, wherein the reference ranges are specific for a specific tumor type. Thus, methods contemplated herein may also be used to characterize a tumor as being immunologically ‘hot’ or ‘cold’.

In another aspect of the inventive subject matter, the inventors contemplate a method of tailoring an immune therapy for a patient having a tumor. The method comprises of obtaining whole transcriptomic sequencing data from a single tumor of the patient; identifying from the whole transcriptomic sequencing data presence and/or activity of immune competent cells in the tumor; identifying from the whole transcriptomic sequencing data expression level of immune checkpoint markers, and correlating the presence and/or activity of immune competent cells, with the expression level of immune checkpoint markers to tailor a tumor treatment for the patient. The step of identifying expression level may comprise determining over-expression or under-expression for the one or more immune regulatory proteins relative to respective reference ranges, wherein the reference ranges are specific for a specific tumor type. The expression level is preferably measured via qPCR or RNAseq. The immune checkpoint marker is annotated as expressed when the quantified expression level exceeds +/−2SD of the reference range. In some embodiments, the expression level is determined by cutoffs defined in The Cancer Genome Atlas (TCGA) expression profiles. The reference ranges are specific for a specific tumor type as classified in ICD10. The immune checkpoint marker is preferably selected from the list comprising of PDL1, PDL2, CTLA4, IDO1, LAG3, and TIM3. In preferred embodiments, the immune checkpoint marker is PDL1. Once an immune therapy is tailored for a patient using the method disclosed herein, the patient may also be treated for the tumor. Treatment could comprise a PDL1 inhibitor therapy for a PDL1-high tumor, or an IDO inhibitor or TIM3 inhibitor therapy for a PDL1-low tumor. In some embodiments, the expression level of immune checkpoint markers may be determined from a cfRNA sample obtained from blood of the patient.

In further contemplated embodiments, disclosed herein is a method of treating a patient having a tumor, comprising administering to the patient a tailored combination of immune therapies. In preferred embodiments, the tailored combination is determined by the steps of: obtaining whole transcriptomic sequencing data from a single tumor of the patient; identifying from the whole transcriptomic sequencing data presence and/or activity of immune competent cells in the tumor; identifying from the whole transcriptomic sequencing data expression level of immune checkpoint markers; and correlating the presence and/or activity of immune competent cells, with the expression level of immune checkpoint markers to tailor a tumor treatment for the patient.

In a further aspect of the inventive subject matter, the inventors have disclosed a method of priming a patient for immune therapy of a tumor, comprising the steps of quantifying or obtaining expression levels for a plurality of somatic-specific single nucleotide variants (SNVs) from paired tumor and normal whole exome sequencing, predicting MHC1 binding affinity for neoepitope peptides resulting from said SNVs, wherein neoepitope binding to MHC1 results in silenced neoepitopes, identifying the patient for priming upon prediction of silenced neoepitopes; and administering the identified patient with epigenetic priming therapy prior to immune therapy. The SNVs may be annotated as expressed if observed in more than two RNAseq reads. The method disclosed herein may further comprise identifying a patient for the priming therapy, comprising: quantifying or obtaining expression levels for a plurality of distinct genes, wherein the distinct genes are associated with respective distinct types of immune cells; determining over-expression or under-expression for each of the distinct genes relative to respective reference ranges, wherein the reference ranges are specific for a specific tumor type; and using the over-expression and/or under-expression of each of the distinct genes to infer activity and/or infiltration by the immune cells in the tumor, and wherein patients with high immune infiltration are identified for the priming therapy. The gene may be PDL1 (CD274). In some embodiments, the patient identified for priming therapy has low PDL1 expression. The immune therapy may comprise treatment with an immune checkpoint inhibitor. Alternatively or additionally, the immune therapy may comprise treatment with at least one of a vaccine composition and an immune stimulatory cytokine.

In another aspect of the inventive concept, disclosed herein is a method of predicting an effective cancer therapy for a patient, comprising: sequencing whole genomic and transcriptomic data to obtain in silico expressed neoantigens and expressed checkpoint markers, and predicting an effective cancer therapy based on the expressed neoantigens and expressed checkpoint markers, wherein a checkpoint therapy is an effective cancer therapy for the patient when the in silico sequencing data comprises (a) expressed neoantigens that are predicted to bind MEW, and (b) expressed checkpoint markers; a combination of a DNA hypomethylating agent and checkpoint therapy is an effective cancer therapy for the patient when the in silico sequencing data comprises neoantigens that are predicted to bind MEW, but binding is suppressed by methylation; and a combination of a HDAC inhibitor and checkpoint therapy is an effective cancer therapy for the patient when the in silico sequencing data comprises neoantigens that are predicted to bind MEW, but binding is suppressed by heterochromatin remodeling. In one embodiment, the DNA hypomethylating agent is 5-aza-2′-deoxycytidine (5-AZA-CdR). In one embodiment, the heterochromatin remodeling agent comprises Trichostatin A (TSA), trapoxin and/or depudesin.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWING

FIG. 1 is an exemplary flowchart of a method according to the inventive subject matter.

FIG. 2 depicts RNAseq expression of genes in the immune cell panel of FIG. 1 in 1037 clinical cases.

FIG. 3 exemplarily depicts immune cell category activation stratified by tissue-type of the tumor.

FIGS. 4A-4H illustrates exemplary immune cell infiltration/activation for specific immune cell types stratified by tissue-type of the tumor.

FIG. 5 is a table listing statistics for each cancer type.

FIG. 6 is an exemplary report showing high/normal/low calls for a specific tumor sample with regard to ICD10, and z-scores, with detailed results provided for each cell type.

FIG. 7 shows exemplary checkpoint expression patterns for various immune related genes stratified by PDL1 expression category.

FIG. 8 depicts exemplary immune-cell activation in PDL1 categories, allowing for a determination as to whether tissue samples are enriched or suppressed in those cell types.

FIG. 9 depicts associations between immune cell presence/activation in tumor cells as a further function of CMS type, MSI status, and sidedness as determined using the methods presented herein.

FIG. 10 shows exemplary results for immune cell enrichment in MSI and MSS groups as determined using the methods presented herein.

FIG. 11 shows exemplary results for various immune markers MSI high and low groups.

FIG. 12 shows exemplary results for correlation of neoantigen load with tumor mutation burden (TMB).

FIGS. 13A and 13B shows exemplary results for correlation of PDL1 expression to TMB/Neoantigen load.

FIG. 14 shows exemplary results for presence of an expressed MHC binder driven by TMB.

FIG. 15 shows exemplary results for a mosaic plot showing contingency table of expressed/non-expressed variants versus predicted binding/non-binding of resulting neoepitopes.

FIGS. 16A, 16B, and 16C shows exemplary results for the subset of patients that more actively silence binding neoepitopes.

FIG. 17 shows exemplary results for precision medicine as disclosed herein.

FIG. 18 shows exemplary results for immune desert setting.

FIG. 19 shows exemplary results for prediction pipeline for cancer treatment.

### DETAILED DESCRIPTION

The inventor has discovered that immune cell signatures can be obtained from a tumor tissue using gene expression signatures that are specific to or at least characteristic for various immune cells. Viewed from a different perspective the inventors conducted single-cell experiments to define gene sets that can differentiate between immune-cell types. By observing expression patterns of those gene sets within a tumor sample, the inventor was then able to make a determination as to whether a tumor tissue sample is enriched or suppressed in those cell types.

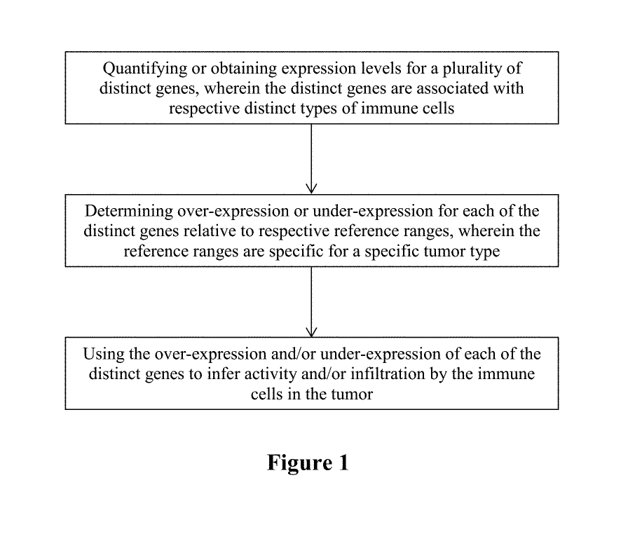

More specifically, based on single cell gene expression analysis of various immune cells, the inventor identified the following genes as being suitable for use in the analyses presented herein: BLK, CD19, CR2 (CD21), HLA-DOB, MS4A 1 (CD20), TNFRSF17 (CD269), which are commonly associated with B cells and are involved in several roles, including generating and presenting antibodies, cytokine, production, and lymphoid tissue organization, CD2, CD3E, CD3G, CD6, which are commonly associated with T cells, various genes associated with helper T cells, including ANP32B (APRIL), BATF, NUP107, CD28, ICOS (CD278) (associated with effector T cells), CD38, CSF2 (GM-CSF), IFNG, IL12RB2, LTA, CTLA4 (CD152), TXB21, STAT4 (associated with T_{H}1 cells), CXCR6 (CD186), GATA3, IL26, LAIR2 (CD306), PMCH, SMAD2, STATE (associated with T_{H}2 cells), IL 17A, IL 17RA (CD217), RORC (associated with T_{H}17 cells), CXCL13, MAF, PDCD1 (CD279), BCL6 (associated with T_{FH} cells), FOXP3 (associated with T_{reg} cells), ATM, DOCKS, NEFL, REPS1, USP9Y (associated with T_{CM} cells), AKT3, CCR2 (CD192), EWSR1 (EWS), LTK, NFATC4 (associated with T_{EM} cells), CD8A, CD8B, FLT3LG, GZMM, MET1, PRF1 (associated with CD8+ T cells), CD160, FEZ1, TARP (TCRG) (associated with T_{γδ} cells), BCL2, FUT5, NCR1 (CD335), ZNF205 (associated with NK cells), FOXJ1, MPPED1, PLA2G6, RRAD (associated with CD56 bright cells), GTF3C1, GZMB, IL21R (CD360) (associated with CD56_{dim} cells), CCL13, CCL17, CCL22 (MDC), CD209, HSD11B1 (associated with dendritic cells), CD1A, CD1B, CD1E, F13A1, SYT17 (associated with immature dendritic cells), CCL1, EBI3, IDO1 (INDO), LAMP3 (CD208), OAS3 (associated with activated dendritic cells), IL3RA (CD123) (associated with plasmacytoid dendritic cells), APOE, CCL 7 (FIC), CD68, CHIT1, CXCL5, MARCO, MSR1 (CD204) (associated with macrophages), CMA1, CTSG, KIT (CD117), MS4A2, PRG2, TPSAB1 (associated with mast cells), CSF3R (CD114), FPR2, MME (CD10) (associated with neutrophils), and CCR3 (CD193), IL5RA (CD125), PTGDR2, (CD294), SMPD3, and THBS1 (associated with eosinophils). These genes were identified to be preferentially or even selectively expressed in certain immune cells (see also e.g., Immunity 39, 782-795, Oct. 17, 2013). FIG. 1 depicts an exemplary flowchart of a method contemplated herein.

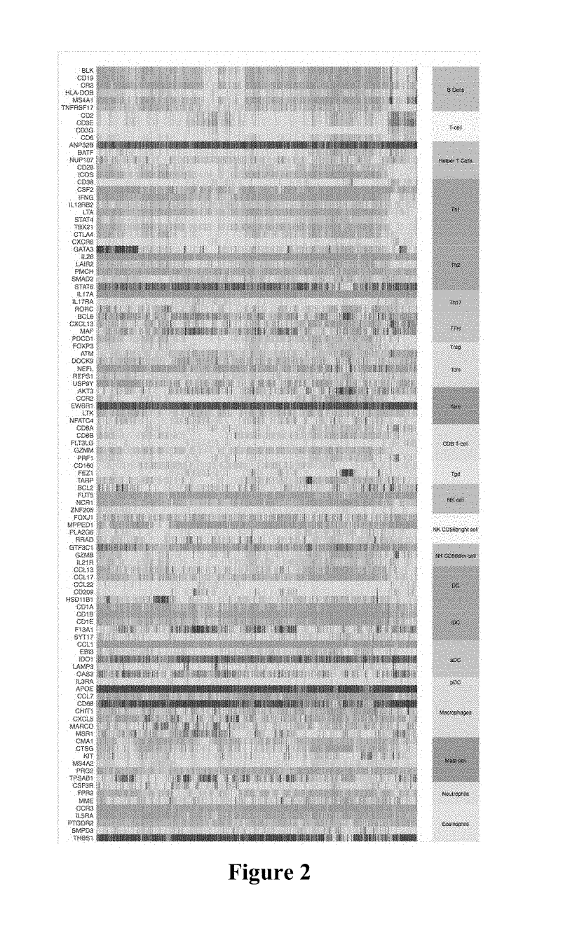

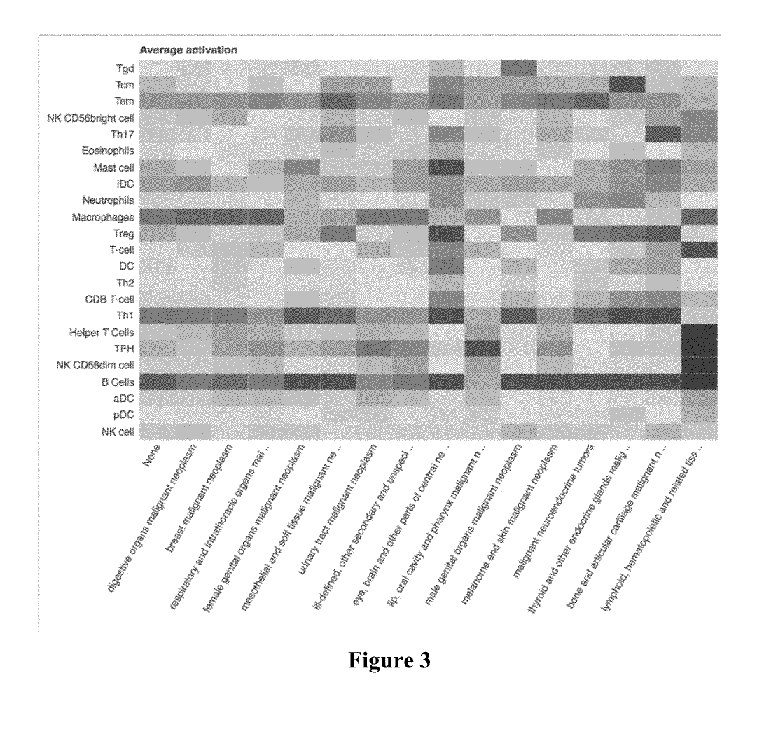

Using these so identified genes, RNAseq analysis was performed on a total of 1037 tumor samples to investigate whether RNA expression levels of these genes would cluster. FIG. 2 depicts an exemplary result for these tumor samples where the rows are ordered by immune cell categories per FIG. 1, and where the columns are ordered by hierarchical clustering using Pearson similarity score. Colors range from blue (log 2[TPM+1]==0) to red (log 2[TPM+1]˜12.5). Notably, when expression of the immune genes for each immune cell type was averaged, and when the average values were correlated with different cancer types, specific signatures became apparent as is exemplarily illustrated in FIG. 3. Here, the heat map shows an average expression for all genes in each immune cell category, split up into reported ICD10 categories (which are representative of tumor classifications). The rows are ordered by hierarchical clustering (using Pearson similarity score), while the columns are ordered from left-to-right by how many samples were annotated for that cancer type. Colors range from blue (avg. log 2[TPM+1]˜0.35) to red (avg. log 2[TPM+1]˜5.0).

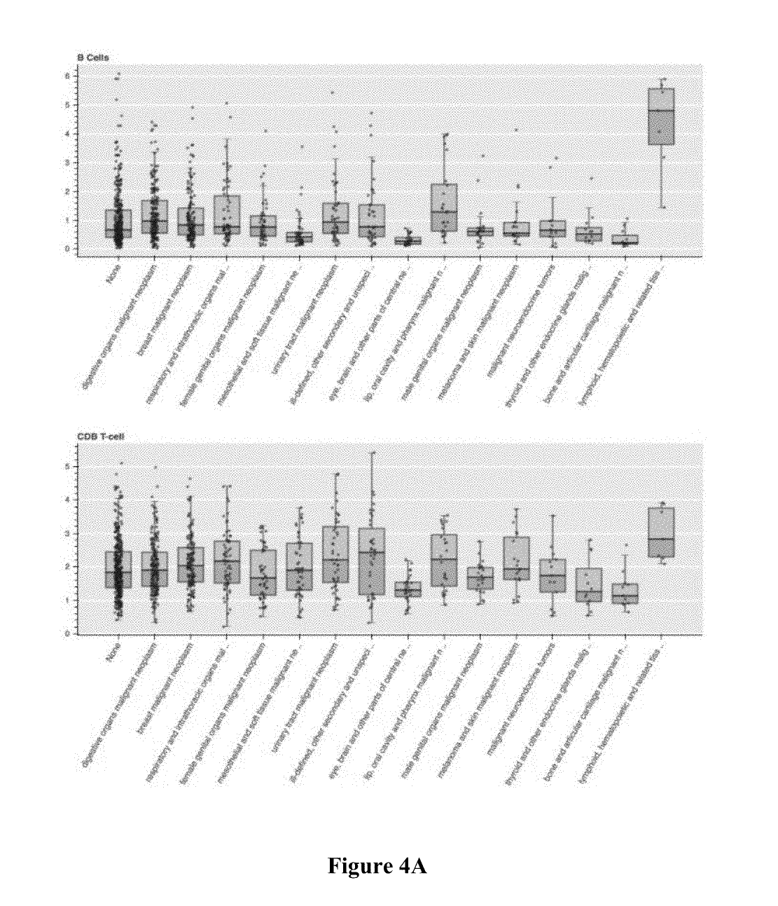

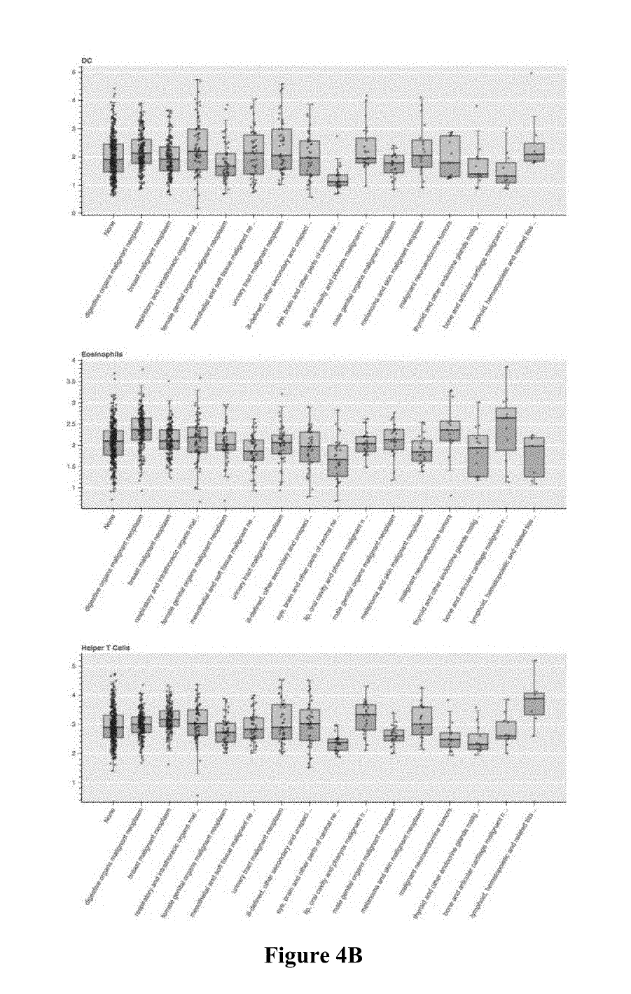

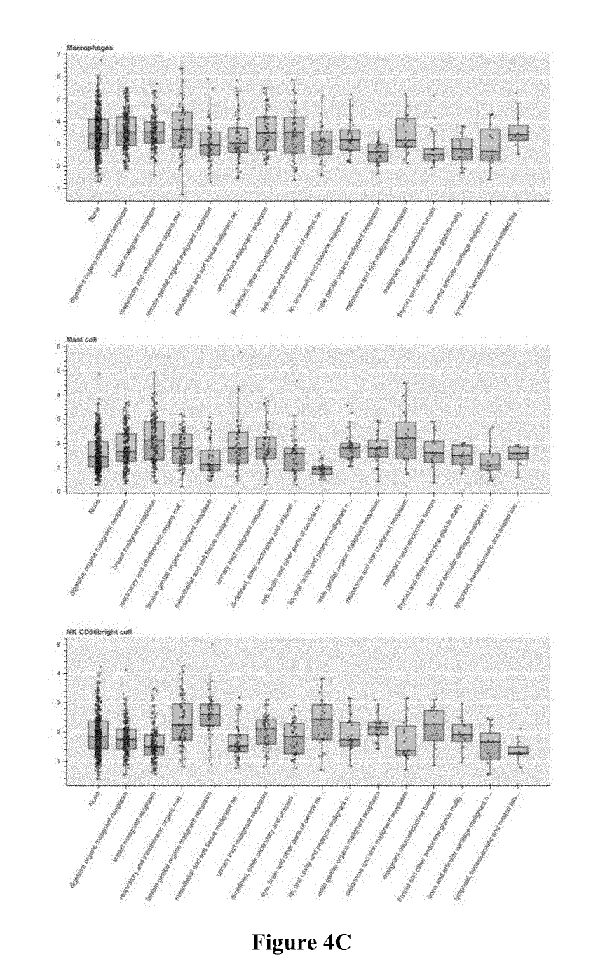

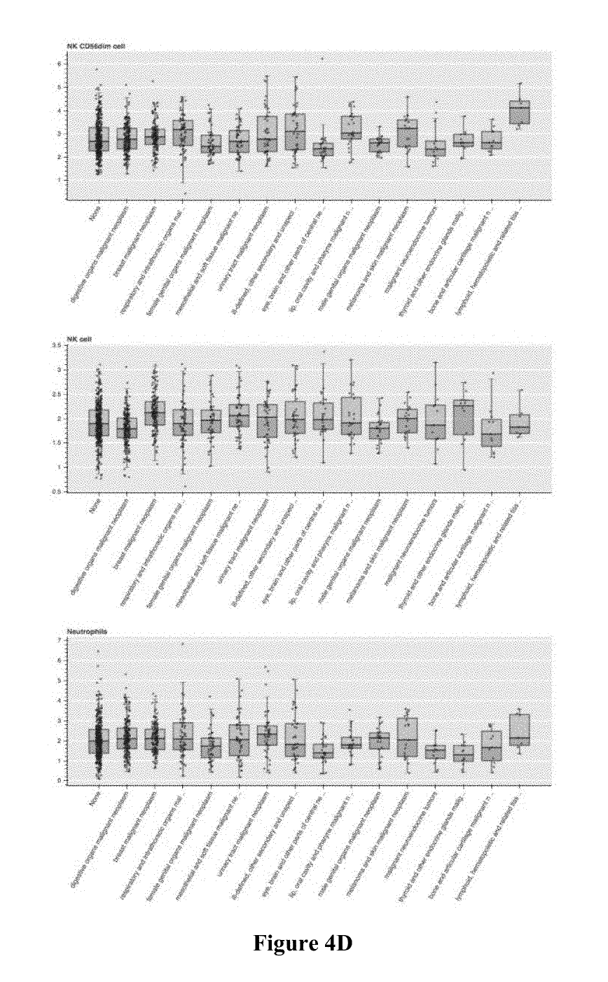

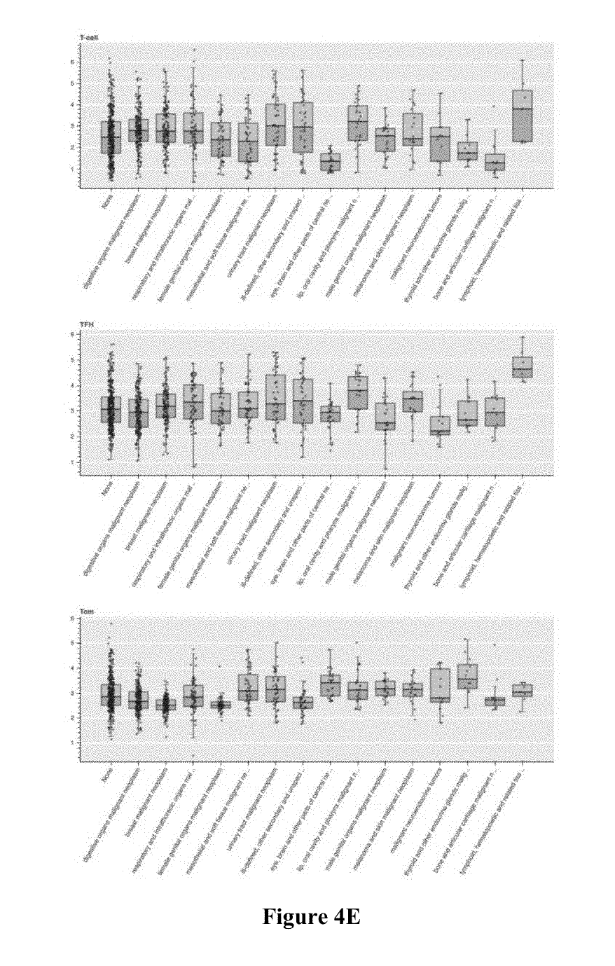

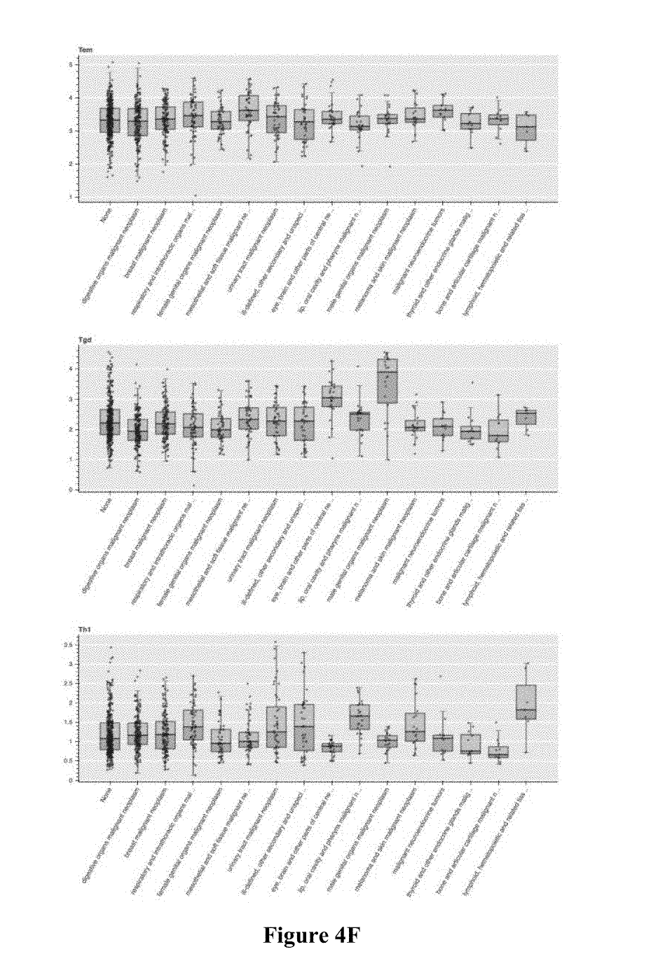

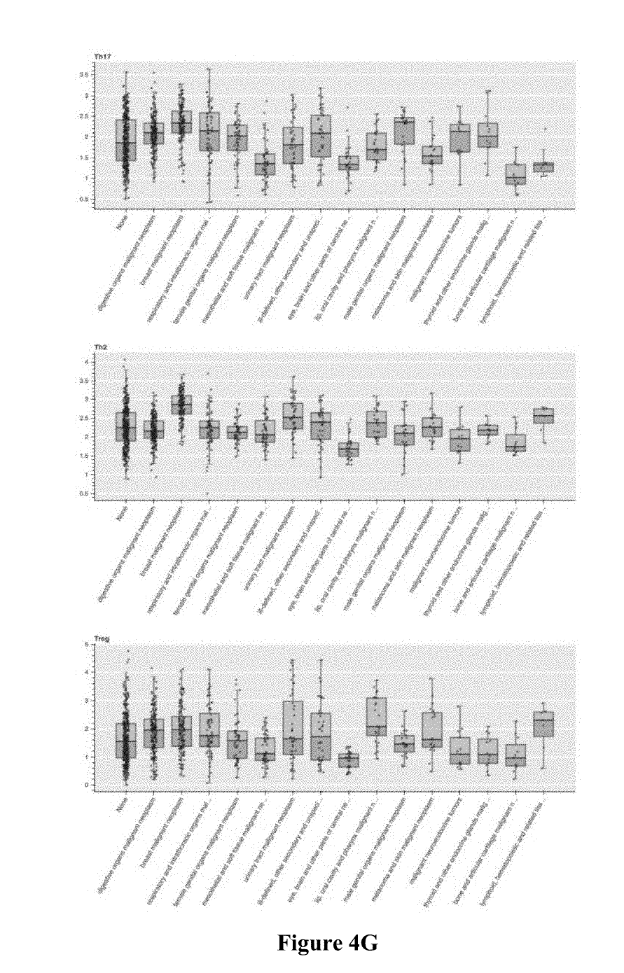

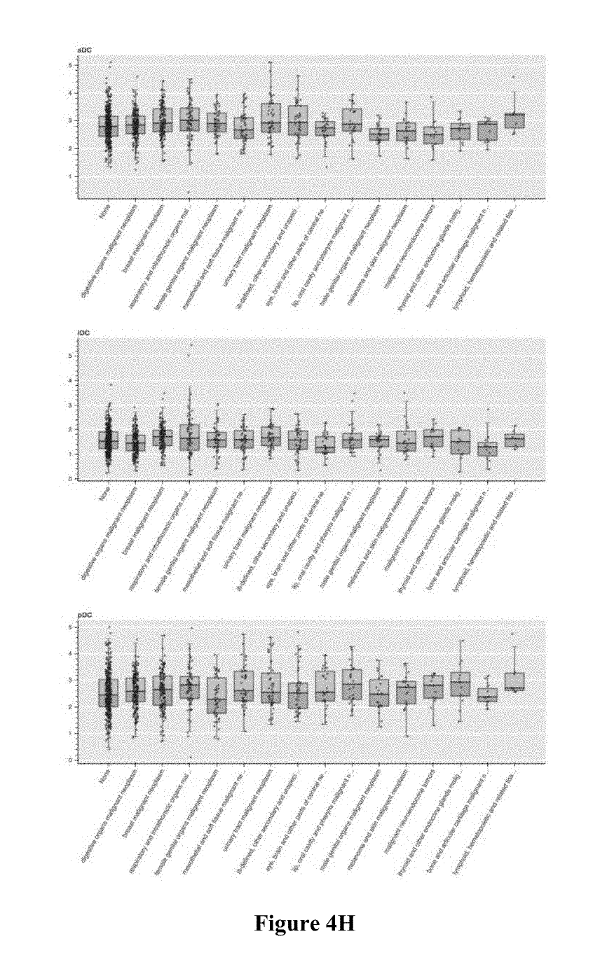

The inventor then investigated whether specific immune cell types would be equally or differentially present or active in different types of tumors. Unexpectedly, as can be seen from the graphs in FIGS. 4A-411, distinct activation patters became evident for the particular immune cell type and cancer involved. More specifically, all RNA expression data were analyzed using log 2[tpm+1] expression for all genes in each immune cell category and split up into reported ICD10 categories. The data points in FIGS. 4A-4H are individual reported cases, boxplots are derived from the category (max z=1.5), and the cancer type categories are ordered from left-to-right by how many samples were annotated for that cancer type. As is readily apparent from the results, the strength of expression for the same genes of a single immune cell type varied significantly among different tumor cell types. Moreover, to a lesser degree the range of expression also varied among different tumor cell types. It should further be appreciated that the diversity in gene expression of a single immune cell type among different tumor types was similarly observed for different immune cell types within the same tumor tissue type. Viewed from a different perspective, gene expression of the above noted genes in immune cells was idiosyncratic with regard to a specific tumor type and type of immune cell.

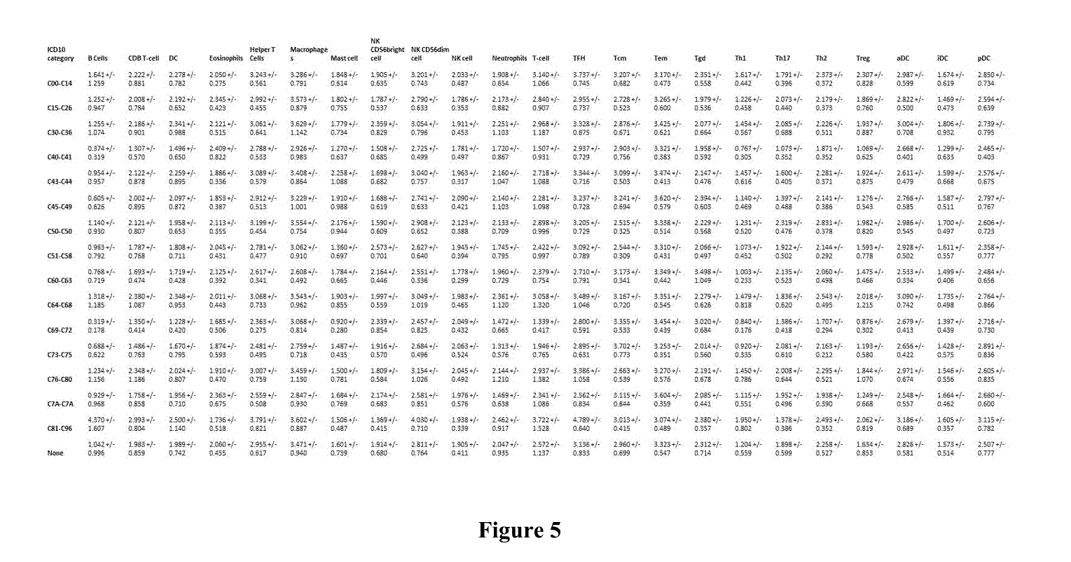

The inventor then employed statistical analysis for the average gene expression of the particular immune cell and cancer type to identify threshold expression levels for the genes in specific immune cells with regard to a specific tumor cell type. Exemplary results are shown in the table of FIG. 5. Here, the mean and standard deviation log 2[tpm+1] for all genes in each immune cell category are listed, and stratified into the reported ICD10 category. Once more, it can be readily seen from the data in FIG. 5 that different immune cell categories had different mean expression rates for the genes specified above and in FIG. 1. Consequently, using such deconvoluted information, these statistics can then be advantageously used to determine over-(>2sd), under- (>−2sd), or normal-activation given a particular tumor tissue type. Thus, it should be appreciated that such quantitative analytic process can be used to correlate gene expression (e.g., as measured by RNAseq) with the presence and/or activity of specific immune cells in the tumor, and with that to infer whether a tumor is immunologically ‘hot’ or ‘cold’.

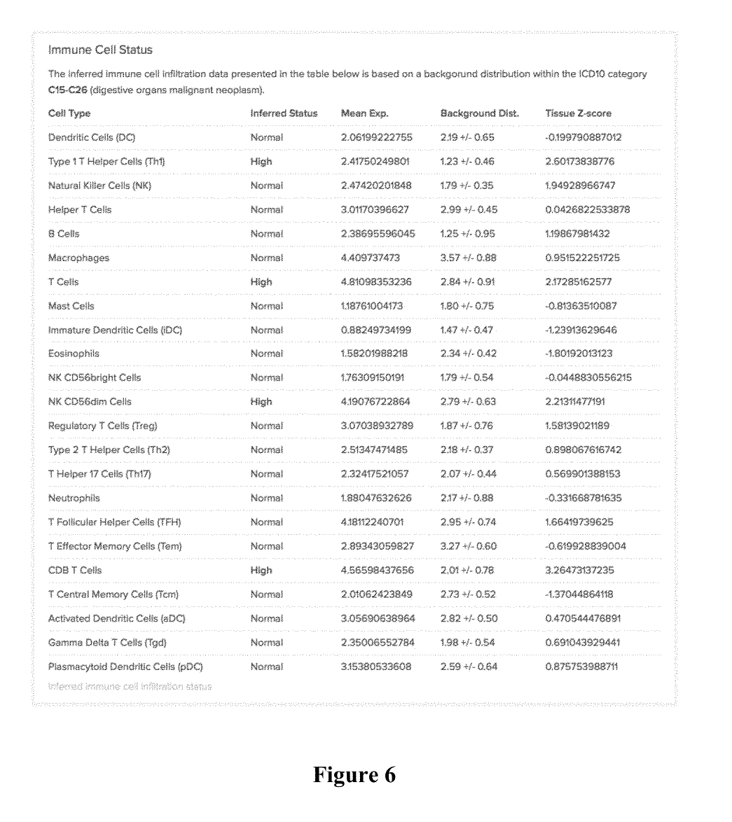

For example, a tumor tissue belonging to ICD10 class C15-C26 (here: digestive organs malignant neoplasm) can be analyzed using RNAseq and gene expression data quantified, using the specific tumor tissue type and the tabulated results of FIG. 5. Based on these results, as is exemplarily shown in FIG. 6, immune cell type status/presence can be readily inferred. In the example of FIG. 6, the tumor sample has higher than normal activity of Th1 cells, T cells, NK cd56dim cells, and CDB T cells. Viewed from a different perspective, it should be recognized that gene expression quantification of specific genes associated with specific immune cells (normalized by tumor tissue type) can be used to infer immune cell infiltration and/or immune cell activation. In this exemplary report format, an inferred status is included that indicates the kind and/or number of types of immune-cell types are elevated (e.g., 4 elevated signatures).

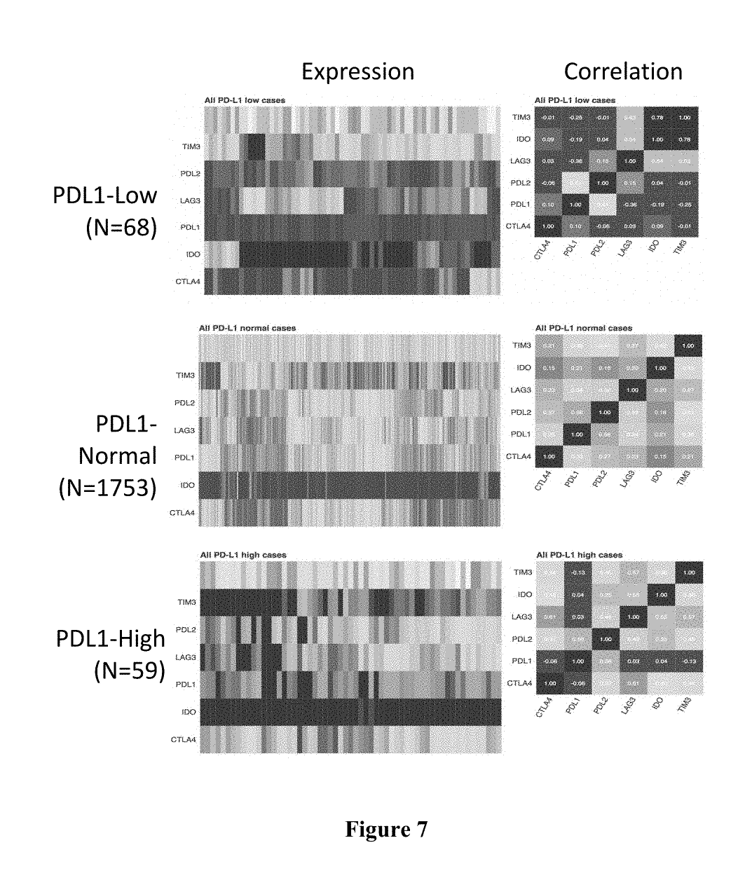

In still further studies, the inventor investigated whether or not immune marker co-expression patterns could be identified, and particularly checkpoint expression patterns and their correlations. For example, the inventors investigated if for a given PDL1 expression level in a tumor as measured by RNAseq any association could be identified with respect to other checkpoint related genes and their expression levels. More specifically, FIG. 7 shows exemplary checkpoint expression patterns. Here, the expression heatmaps are log 2[tpm+1] scale with (blue=0, red>=5), and the colors at the top indicate the different ICD10 cancer types. Expression heatmaps are ordered by Euclidean distance, and the correlation plots are Pearson correlations (blue=0, red>=0.75). Notably, and in contrast to the immune gene expression patters discussed above, there was an apparent lack of significant tissue-dependent expression of immune checkpoints as can be taken from the unclustered appearance of the cancer type color indicators. As expected, however, PDL1 and PDL2 expression was moderately correlated.

Additionally, it was also observed that IDO and TIM3 had relatively high expression, particularly in the absence of PDL1 or in cases with low PDL1 expression. Expression levels of IDO and TIM3 were also highly correlated (R=0.78) when PDL1 is under-expressed, and that relationship seemed to be inversely proportional to PDL1 expression. LAG3 was also correlated with IDO and TIM3 in a low PDL1 setting, however, this relationship was not clear as PDL1 increased. Consequently, the data suggest that PDL1 itself is sufficient as a primary driver of immune suppression (as seen in the PDL1-high correlation plot), however when PDL1 is low there may be some differential role for IDO and TIM3.

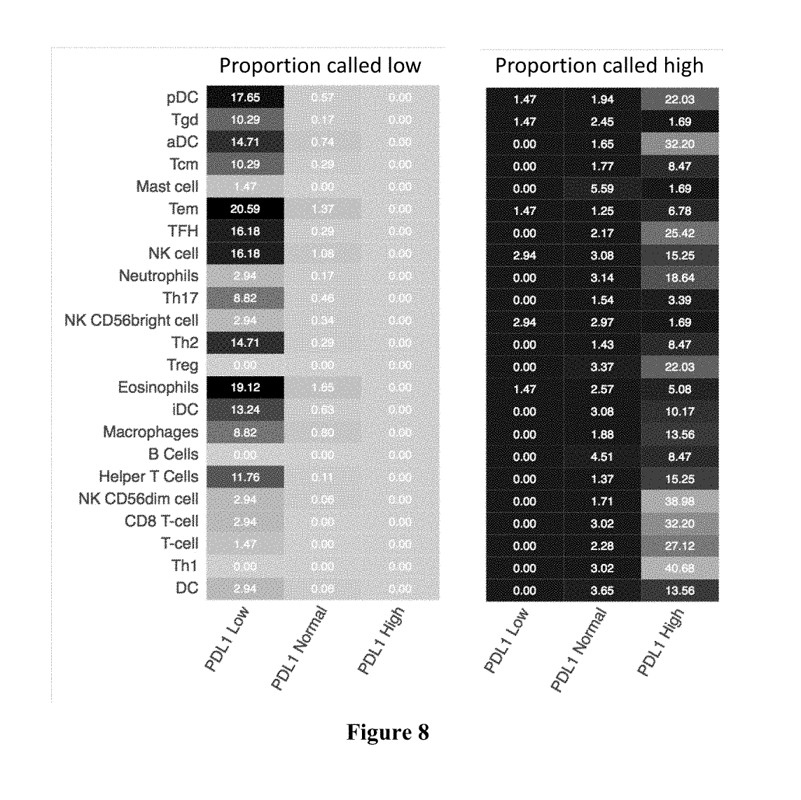

When further investigating the role of PDL1 with respect to immune cell categories as noted above, the inventor discovered that the PDL1 high group is enriched for multiple immune-cell types, including multiple kinds of T-cells & T-helper cells as can be seen in FIG. 8, right plot (depicting relative over-representation). Thus, especially in conjunction with the results of the checkpoint expression patterns seen in FIG. 7, it appears that PDL1 expression is probably sufficient to evade these systems. On the other hand, in the PDL1 low group CD8 T-Cells, T-Cells, and Th1 cells are not significantly under-represented, however most other category of immune cells are including NK, and memory T cells as can be seen in FIG. 8, left plot (depicting relative under-representation). Taken with the results of FIG. 7, the expression data are indicative that IDO and TIM3 have a strong role in regulating memory T cells.

Therefore, it should be appreciated that immune cell specific gene expression analysis can be used in predictive analysis of immune therapy, particularly for immune therapy targeting the PD1/PDL1 axis. On the other hand, alternative immune therapy targeting IDO and/or TIM3 may also be indicated where the tumor tissue is PDL1 low.

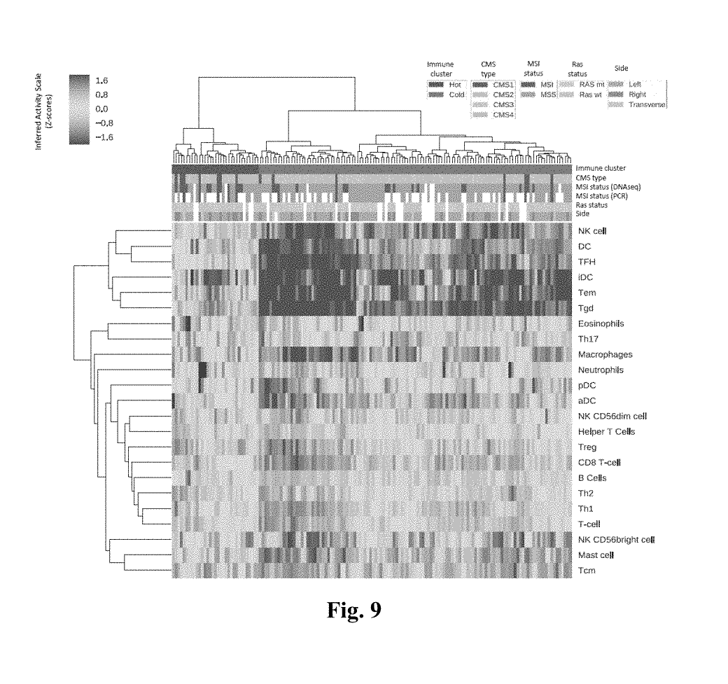

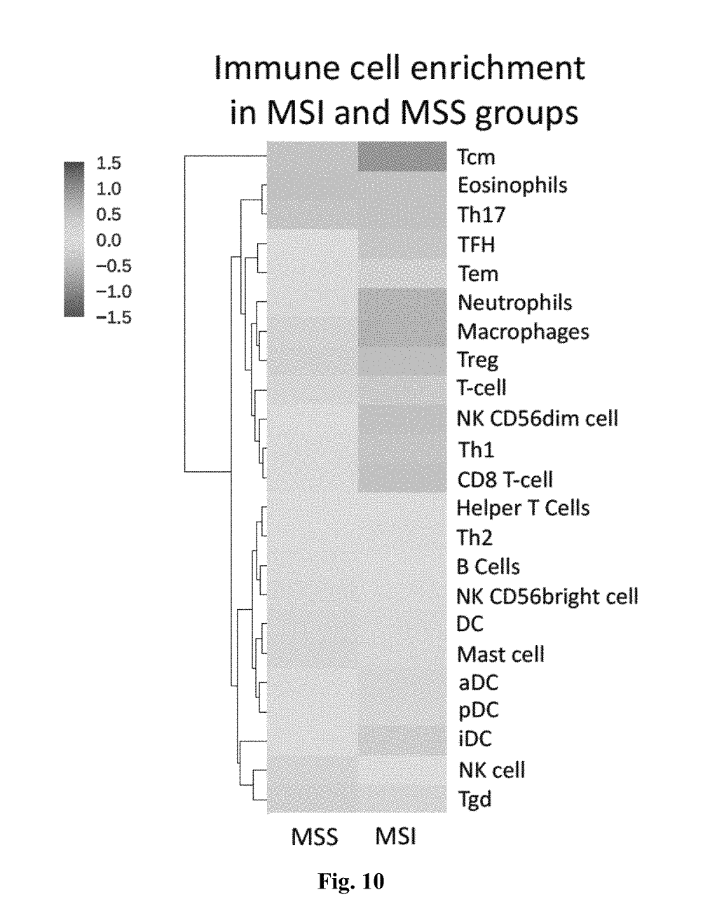

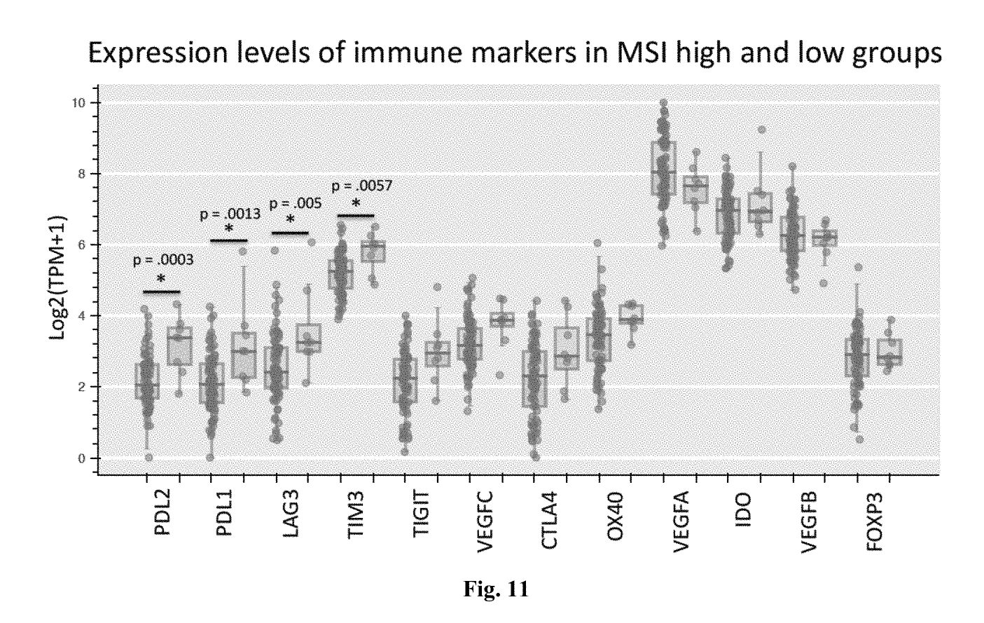

In still further experiments, immune status was also correlated with MSI status on a total of 152 colorectal cancer tumor samples. Tumor/normal-paired DNAseq (WGS or WES) and deep RNAseq was performed and MSI-status was determined by both PCR and WGS/WES profiles. CMS types, checkpoint expression, and immune-infiltration deconvolution were calculated upon RNAseq data using above sequences, and significant enrichment for MSI, immune status, CMS types, and clinical covariates were analyzed. FIG. 9 depicts exemplary results for the analysis. As can be seen from FIG. 9, clustering of immune expression bifurcated well in to hot and cold tumors. Moreover, significant association was found between CMS1, MSI, transverse sides, and being immunologically hot. Conversely, CMS2 was found to be significantly MSS, left-sided, and immunologically cold. Thus, CMS1 tumors that are immunologically hot appear to be treatable with immune checkpoint inhibitors. In yet another set of experiments, total of 521 GI patients with deep whole exome sequencing (WES) of tumor and blood samples, and whole transcriptomic sequencing (RNA-Seq) (˜200M reads per tumor) were available for this analysis from a commercial database. Variant calling was performed through joint probabilistic analysis of tumor and normal DNA reads, with germline status of variants being determined by heterozygous or homozygous alternate allele fraction in the germline sample. MSI was determined via a CLIA LDT based on NGS data at microsatellite sites. Notably, higher immune signaling was observed in MSI high tumors, and some MSI samples showed high CD8 T-cells enrichment. Moreover, and as observed before, TIM3 and LAG3 were expressed at higher levels in MSI high samples. Typical results are depicted in FIGS. 10 and 11. As can be seen from FIG. 10, enrichment of various immune cell types in the two MSI groups is shown. The brighter the red color is the larger the enrichment. Likewise, FIG. 11 illustrates the expression levels of various immune markers in the two MSI groups. Here, PDL2, PDL1, LAG3, and TIM3 are statistically significantly differentially expressed. TIM3 presents an interesting potential therapeutic target.

It should still further be appreciated that contemplated methods and analyses may also be useful in determination of suitable treatment where location may provide a contributing factor. For example, the inventor discovered that upper and lower GI tumors are distinct in their tolerated immune cell infiltration. Immune therapies should therefore be tailored based on location to take advantage of the innate immune apparatus present. Specifically, upper GI cancers appear especially fit for checkpoint therapy despite having lower average TMB.

In one preferred aspect, the inventors have found that profiling the tumor and associated microenvironment can help tailor rational combinations of immunotherapeutic strategies. Thus in one embodiment, disclosed herein is a method of tailoring an immune therapy for a patient having a tumor, wherein whole transcriptomic sequencing data is obtained from a single tumor of the patient, the whole transcriptomic sequencing data is processed to identify the presence and/or activity of immune competent cells in the tumor, as well as to identify expression level of immune checkpoint markers. The presence and/or activity of immune competent cells and the expression level of immune checkpoint markers are then correlated to tailor a tumor treatment for the patient. The expression profile of PDL1 was investigated in various types of cancers, such as breast cancer, lung cancer, colon cancer, pancreatic cancer, ovarian cancer, brain cancer and prostate cancer. It was found that when the expression level of PDL1 is lower than normal for that specific cancer (PD-L1-low category), the tumor cells were especially deprived of memory T cells and eosinophils. Thus, in these cases, it is ideal to use of indoleamine 2,3-dioxygenase (IDO) or T-cell immunoglobulin mucin-3 (TIM3) directed therapies.

It should be appreciated that IDO is an inducible enzyme that catalyzes the rate-limiting first step in tryptophan catabolism. IDO causes immunosuppression through breakdown of tryptophan in the tumor microenvironment and tumor-draining lymph nodes. The depletion of tryptophan and toxic catabolites renders effector T cells inactive and dendritic cells immunosuppressive. The inventors have found that when a specific cancer in a specific patient has low expression of PDL1, IDO may be a preferable drug target. In these cases, IDO inhibition can delay tumor growth, enhance dendritic cell vaccines, and synergize with chemotherapy through immune-mediated mechanisms.

The inventors have also found that TIM3 directed therapies are preferably used in the patients whose tumors express lower than normal levels of PDL1. TIM-3 is a cancer immune checkpoint, inhibiting anti-tumor immunity. The inventors have found that when a specific cancer in a specific patient has low expression of PDL1, TIM-3 would be a preferable drug target. TIM-3 is expressed in a variety of immune cells, including T cells, regulatory T cells (Tregs), dendritic cells (DCs), B cells, macrophages, nature killer (NK) cells, and mast cells, and therefore it is a favorable target for a variety of cancers.

Viewed from another perspective, this work indicates that PDL1 expression by RNAseq is a biomarker for anti-PD1 therapy (i.e. a CDx), and superior to tumor mutational burden (TMB). This work is also proposes the use of an immune-profile of a patient as a diagnostic for the disease and treatment thereof. Finally, this disclosure provides combinations that may be efficacious in immune therapy of cancer patients. For example e.g. PD-1 monotherapy is efficacious for PD1+ patients, while IDO1i+LAG3i/TIM3i therapy is efficacious for PD1-patients.

In another aspect, the inventors have found selective silencing of MHC-binding neoepitopes to avoid immune surveillance. The inventors identified somatic-specific single nucleotide variants (SNVs) from paired tumor/normal whole-exome sequencing (WES). The SNVs were annotated as expressed if observed in more than two RNAseq reads. MHC1 binding affinity for 9-mer neoepitope peptides resulting from said SNVs were predicted using NetMHC within presented HLA-types. The inventors found that several neoepitopes that were predicted to bind MHC1 were silenced or not expressed. The silencing rate increased in patients with high inferred immune infiltration. A further silencing enrichment was observed in patients displaying high immune activity but low PDL1 expression. Based on these results, the inventors have found that patients with tumor infiltrating lymphocytes and silenced neoepitopes would benefit from epigenetic priming therapy prior to immune checkpoint inhibition therapy. Thus, in one embodiment, the inventors have disclosed a method of priming a patient for immune therapy of a tumor, comprising: quantifying or obtaining expression levels for a plurality of somatic-specific single nucleotide variants (SNVs) from paired tumor and normal whole exome sequencing; predicting MHC1 binding affinity for neoepitope peptides resulting from said SNVs, wherein neoepitope binding to MHC1 results in silenced neoepitopes, identifying the patient for priming upon prediction of silenced neoepitopes, and administering the identified patient with epigenetic priming therapy prior to immune therapy.

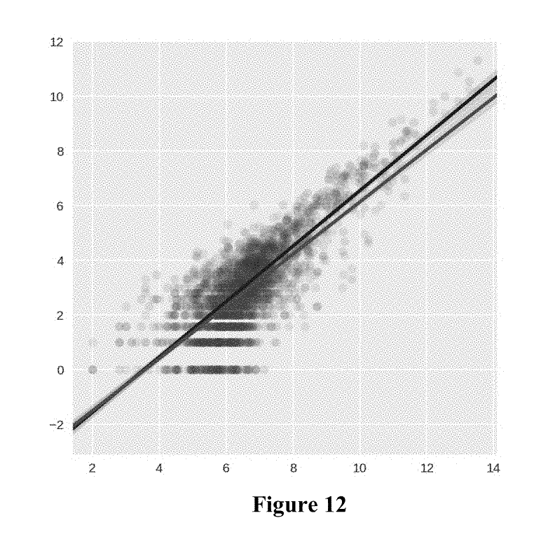

The differential immunogenicity in non-expressed variants is further illustrated in FIGS. 12-16. In one embodiment, the inventors dataset comprised 1395 clinical cases with at least one variant across 39 cancer types with T/N/R sequencing. Somatic-specific non-synonymous variants were identified in 16,403 genes (i.e. whole genome). Patients were HLA-typed by DNAseq. All possible 9-mer neoepitope peptides containing a somatic-specific alteration were tested for binding affinity to MHC class 1 using NetMHC. Strongest binders for each HLA-allele presented by patients were kept for analysis. RNAseq read depth >=2 was used to determine if neoepitopes were expressed or silenced (avg. depth ˜100×). No significant RNAseq depth differences were observed in genes that had silenced neoepitopes, reducing the risk that effects seen are due to coverage bias. In total 147,015 HLA/predicted-affinity combinations were analyzed for expression.

In one embodiment, as illustrated in FIG. 12, the inventors have disclosed that neoantigen load is unlikely to improve checkpoint response prediction accuracy over TMB. Moreover high TMB may be driven by presence of non-expressed variants. Neoantigen-load (i.e. number of predicted binders) was found to be highly correlated with TMB (Spearman Rho=0.75, p=1.3e-255). The correlation was especially strong within the TMB-high category (Rho=0.76) as opposed to in the TMB-low category (Rho=0.63). Even when only analyzing non-expressed variants, neoantigen-load was highly correlated with TMB (Spearman Rho=0.699, p=2.3e-196). Because neoantigen load is functionally equivalent to TMB especially in the TMB-high category, it is unlikely to improve checkpoint response prediction accuracy over TMB. Furthermore, having high TMB, and even high neoantigen-load, can be driven by presence of non-expressed variants.

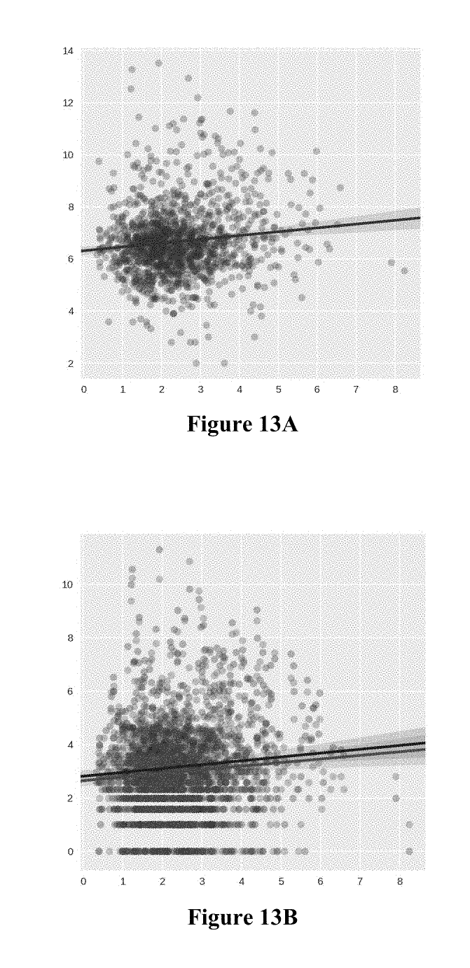

Furthermore, as illustrated in FIGS. 13A and 13B, PDL1 expression is an independent biomarker from TMB/neoantigen-load. There is little correlation between PDL1 expression and TMB (Rho=0.099), neoantigen-load (Rho=0.049), or expressed neoantigen-load (Rho=0.071).

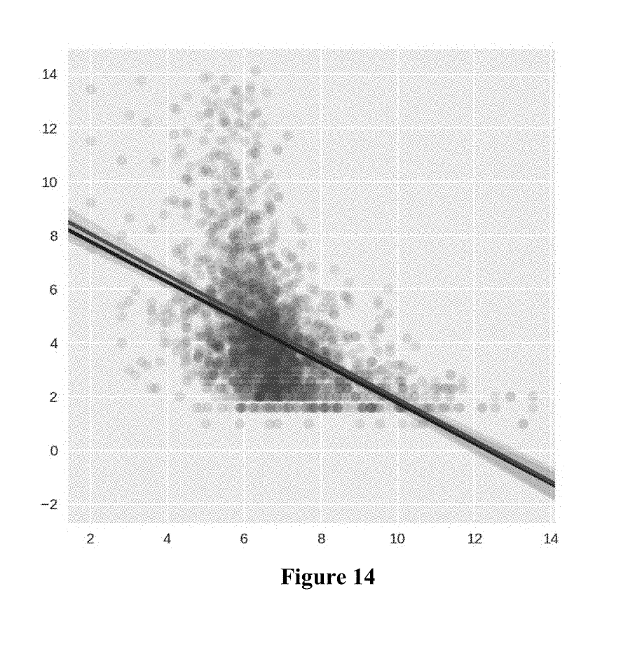

In one embodiment, the inventors sought to determine whether presence of an expressed MHC binder is driven by TMB. FIG. 14 illustrates a plot of the tightest binder present in a patient vs. their TMB (Rho=−0.48, p=9.6e-79), in both expressed and non-expressed variants. (Note lower affinity score means tighter binding). It was found that the 17% of patients that fall into the TMB-high category all express at least one weak-binding neoepitope, and the majority express a strong binder (Table 1 and Table 2). However, 64% of all patients were within the TMB-low category but express at least one strong binder. These patients may respond to checkpoint therapy. Thus, the inventors determined that High TMB is therefore an insufficient biomarker to identify ICT candidates.

Within the TMB-low category, 36 patients (˜3.2%) were identified that express non-binding neoepitopes and do not express potentially binding ones (i.e. selective silencing). Of those, half (i.e. 18 patients) silence expression of a strong-binder. Thus, in one embodiment, the inventors found that selective silencing of MHC-binding neoepitopes is a factor in resistance to ICI therapies.

TABLE 1

| | No WB | Expressed |
|---|---|---|
| | present | WB present |
| Low TMB | 68 | 1063 |
| High TMB | 0 | 232 |

OR = inf,
p = 2.16e−6

TABLE 2

| | No WB | Expressed |
|---|---|---|
| | present | WB present |
| Low TMB | 68 | 1063 |
| High TMB | 0 | 232 |

OR = 16.6,
p = 2.8e−18

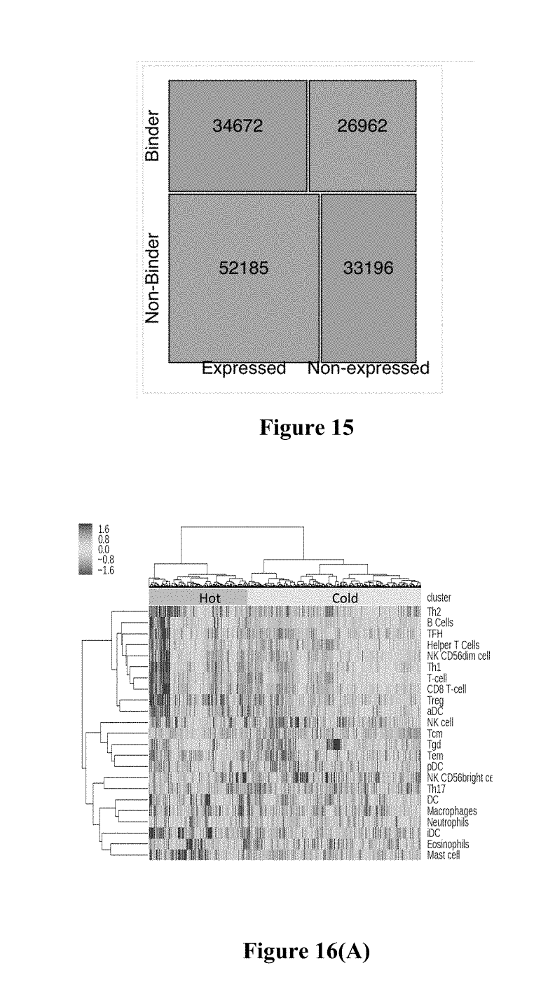

Surprising, the inventors also found systematic silencing of MHC-binding neoepitopes. The mosaic plot in FIG. 15 depicts the contingency table of expressed/non-expressed variants vs. predicted binding/non-binding of resulting neoepitopes. 40.9% of exome-wide somatic-specific variants had low RNAseq support. Surprisingly, this is higher than the rate of ˜18% previously reported within small set of highly curated oncogene/tumor suppressor variants. Across all patients (even those expressing checkpoints) there is a significant silencing of neoepitopes with predicted <500 nM binding affinity (OR: 1.22, p=2.35e-78 One-sided Fishers exact test)

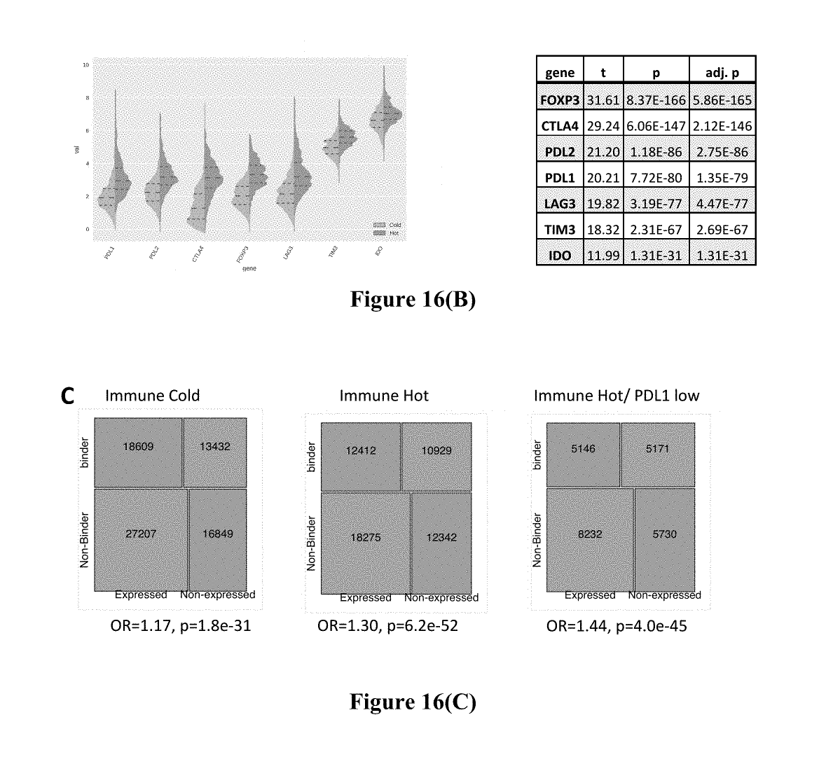

In one embodiment, the inventors also found that patients with active T-cells but lower checkpoint expression would be good candidates for epigenetic priming (e.g. HDACi/5-aza) followed by ICI, as these patients more actively silenced binding neoepitopes. FIG. 16A depicts inferred immune activity as calculated for each patient based on expression of 122 key immune-specific genes (Bindea et al. 2013). Hot (˜34%) and Cold (˜64%) immune activity was assigned according to groupings found using unsupervised clustering (green and yellow above the heatmap). FIG. 16B depicts key targetable checkpoints that were shown to be significantly overexpressed in hot vs. cold tumors. This suggests hot tumors are producing immune-activating neoepitopes, and that immunity is being actively suppressed. FIG. 16C Silencing of potential binders was found to be more enriched in immune-hot than immune cold tumors (OR=1.3 vs. 1.17), and at a higher rate than the background population (1.22). Specifically those with active immune infiltration but low PDL1 expression have the highest observed silencing of binding neoepitopes (OR=1.44)

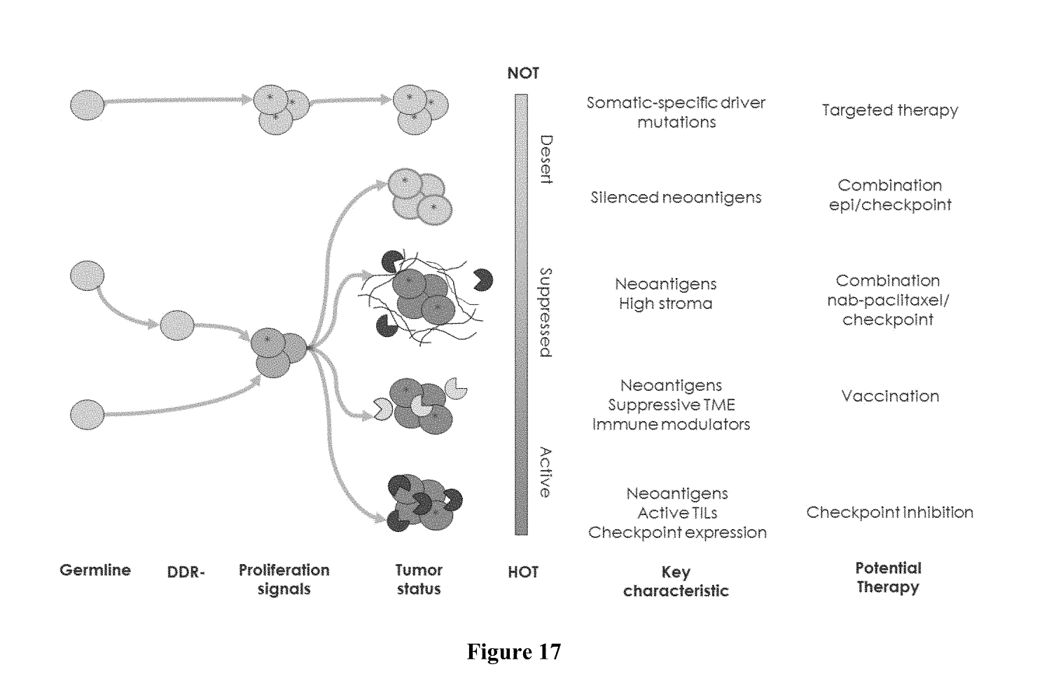

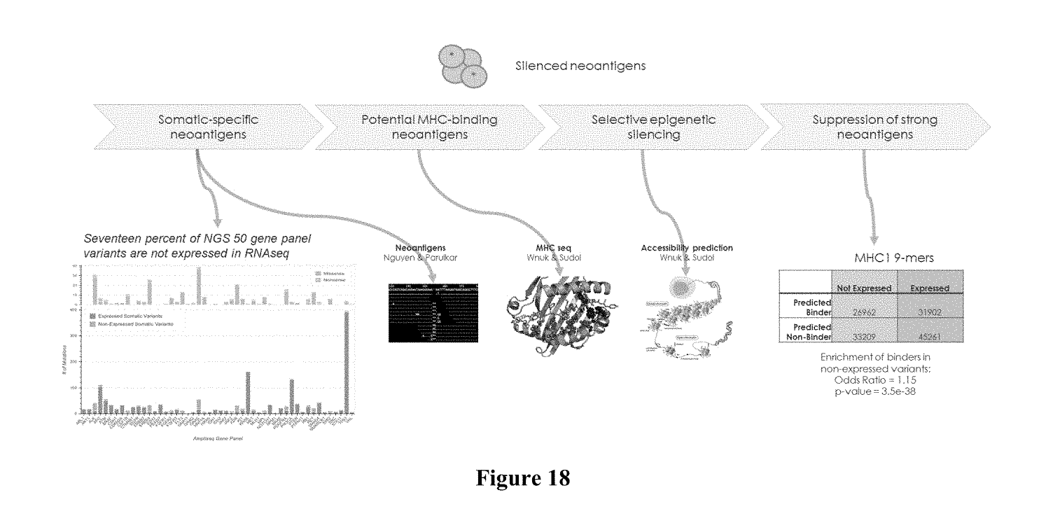

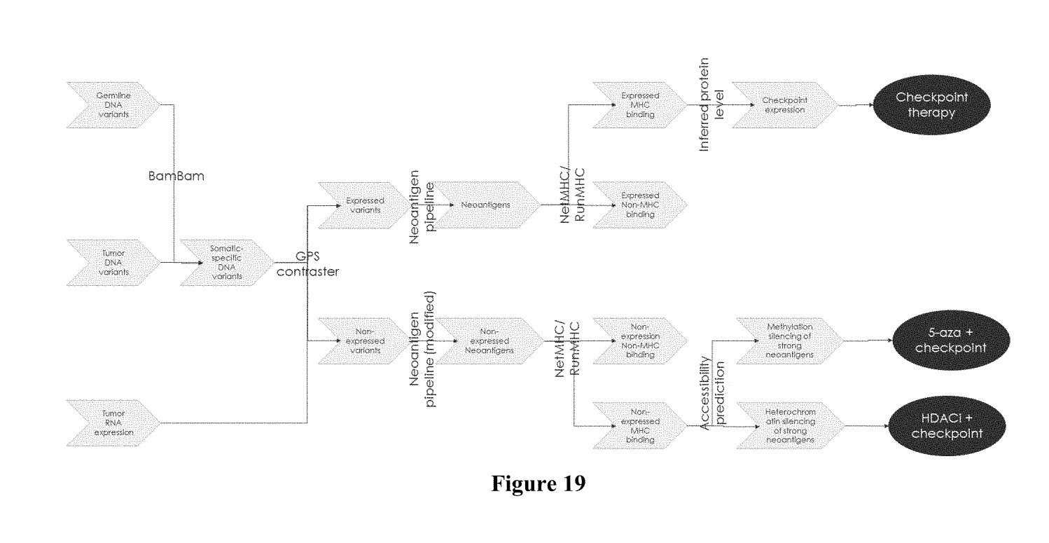

A method of predicting which immune therapy would be suitable for a patient is depicted in FIGS. 17-19. If patients are expressing neoantigens that are predicted to bind MHC and are expressing checkpoint (PDL1 etc.) they are canonical cases for checkpoint (pembro, ipi etc.). If patients have variants that should bind but are not expressed, they may be primed to re-express strong neoantigens. Prediction of whether a patient is in this category may be done by using a modified neoantigen pipeline that assesses binding of peptides that would result from non-expressed variants. Further, DNA-accessibility prediction may be able to predict if the specific neoantigens that should bind MHC are suppressed by methylation or chromatin remodling. In one embodiment, the former would get an anti-methylation drug (5-aza), the latter would get an HDAC inhibitor, for effective treatment of the tumor.

Thus, in one embodiment, the inventive subject matter comprises a method of predicting an effective cancer therapy for a patient, comprising sequencing whole genomic and transcriptomic data to obtain in silico expressed neoantigens and expressed checkpoint markers. Prediction of the effective cancer therapy is done based on the amount of expressed neoantigens and checkpoint markers. For example, a checkpoint therapy is predicted to be effective when the in silico sequencing data comprises expressed neoantigens that are predicted to bind MEW, and expressed checkpoint markers. A combination of a DNA hypomethylating agent and checkpoint therapy is predicted to be effective when the in silico sequencing data comprises neoantigens that are predicted to bind MHC, but binding is suppressed by methylation. In this case, the DNA hypomethylating agent may be 5-aza-2′-deoxycytidine (5-AZA-CdR). A combination of a HDAC inhibitor and checkpoint therapy is predicted to be effective when the in silico sequencing data comprises neoantigens that are predicted to bind MEW, but binding is suppressed by heterochromatin remodeling. All pharmaceutically acceptable heterochromatin remodeling agents are contemplated; non limiting examples being Trichostatin A (TSA), trapoxin and/or depudesin.

### Examples

### Example 1: Co-Expression Patterns of Immune Checkpoint Molecules in Relation to PD-L1 Expression

Targeting immune checkpoints has led to clinical benefit across a variety of tumor types, and employing combinations has enhanced response rates even further. In one embodiment, the inventors have now found that profiling the tumor and associated microenvironment can help tailor rational combinations of immunotherapeutic strategies.

Whole transcriptomic sequencing (RNA-Seq; ˜200×10⁶ reads per tumor) of 1,880 unselected clinical cases was performed (NantHealth; Culver City, Calif.). Cases reflected 38 distinct histologies including but not limited to breast (17.8%), colon (9.5%), lung (7.9%), pancreatic (6.5%), ovarian (5.4%), brain (4.9%) and prostate cancer (2.7%). Cases were categorized as PD-L1-low, PD-L1-normal and PD-L1-high by cutoffs defined in TCGA expression profiles. Expression and co-expression of 6 checkpoint markers (PD-L1, PD-L2, CTLA4, IDO1, LAG3 and TIM3) were analyzed for tissue-specific enrichment and within PD-L1-defined categories. Immune-cell infiltration was estimated using RNA deconvolution based on known immune cell marker genes.

The inventors found that checkpoint expression did not cluster in a tissue-dependent manner. PD-L1 shows no significant co-expression pattern with any of the analyzed checkpoint markers aside from its ortholog PD-L2 (R=0.77; P=1.9×10⁻²⁸⁵). Within the PD-L1-low category, IDO1 and TIM3 had relatively high expression and were highly correlated with each other (R=0.81; P=4.6×10⁻¹⁷). The PD-L1-low category was especially deprived of memory T cells and eosinophils. Within the PD-L1-high category, overall expression of all checkpoint markers was higher. Amongst PD-L1 high patients, CTLA4 expression was highly variable (mean 2.5±1.1; log₂[TPM+1]) and lacked correlation with PD-L1 (R=−0.09). In contrast, while LAG3 also had variable expression in the PD-L1-high setting, it was strongly correlated with CTLA4 (R=0.79, P=7.4×10⁻¹⁴). The PD-L1-high category was especially enriched for Th1, NK CD56_{dim}, and CD8 T-cells.

Thus, in one embodiment, the inventors found that high and low PD-L1 expression in the tumor and adjacent microenvironment are associated with variations in key checkpoint molecules. Low expression of PD-L1 may be an ideal setting for use of DO- or TIM3-directed therapies.

### Example 2: Evidence for Selective Silencing of WIC-Binding Neoepitopes to Avoid Immune Surveillance

Overall response rates to immune checkpoint inhibition (ICI) are <50% even in TMB-high patients (e.g. Checkmate-227), suggesting other mechanisms of immune escape exist beyond expressing checkpoints. At least 18% of somatic-specific exonic DNA variants are not expressed into mRNA, yet the selection criteria for which variants to silence remains unclear. The inventors determined whether immunogenicity of variants factors into their suppression.

Somatic-specific single nucleotide variants (SNVs) were identified from paired tumor/normal whole-exome sequencing (WES), and annotated as expressed if observed in >=2 RNAseq reads. MHC1 binding affinity for 9-mer neoepitope peptides resulting from said SNVs were predicted using NetMHC within presented HLA-types. Cases with >200 non-synonymous exonic mutations were designated as TMB-high in accordance with Rizvi et al, 2015. Tumor immune activity was inferred by RNAseq expression of 6 checkpoint/TME markers, as well as by estimating immune infiltration using RNAseq deconvolution of immune genesets. Significant associations between TMB, neoantigen-load, expressed neoepitope binding affinities, and immune activity were analyzed.

Within a clinical database of 1.363 cases with T/N/R. sequencing, a total of 147,015 potential neoepitopes were identified. A small but significant enrichment was observed for silencing neoepitopes that are predicted to bind MHC1 (OR 1.22, p=2.4e-78 one-sided Fishers test). The silencing rate was similar between the 17% of patients with high TMB vs others, but was increased in 35% of all patients with high inferred immune infiltration. (N==490, OR=1.30, p=1.8e-31). A further silencing enrichment was observed in 19% of all patients displaying high immune activity but low PDL1 expression (N=263, OR=1.44, p=4.0e-45).

The inventors observed significant preferential silencing of MHC binding neoepitopes. Specifically, when tumor infiltrating immune cells are activated, silencing neoepitopes may be an alternative to checkpoint expression for avoiding an immune cascade. Patients with TILs and silenced neoepitopes may benefit from epigenetic priming therapy prior to ICI therapy.

### Example 3: PDL1 Landscape in Checkpoint Inhibitor Ineligible Patients by IHC and cfRNA

Immune checkpoint inhibitors (ICT) are indicated in patients with PDL1+ IHC and restricted to certain histologies. Other tumor histologies express PDL1 at various frequencies with no established threshold for therapeutic efficacy. In one embodiment, the inventors evaluated traditionally ICI non-indicated histologies by IHC and cfRNA to determine potential therapeutic thresholds.

A total of 97 pts (cancer of unknown primary [N=10], appendix [N=6], bile duct [N=4], colorectal cancer [N=25], esophageal [N=6], ovary [N=7], pancreas [N=15] other [N=24]) with WIC (Dake 76 22C3, 21 SP142) and cfRNA for PDL1 were available for analysis. cfRNA was a random draw not matched with the time of tissue collection and performed by qPCR. A cutoff of >1.5× for PDL1 normalized to beta actin was defined as PDL1+ in cfRNA. IHC >10% was used as the threshold for IHC+.

Most patients were PDL1− by IHC: 90/97 (93%), PDL1 TPS: 0% (N=72), 1% (N=7), 5% (N=8), 10% (N=3), >10% (N=3), >50% (N=4). No difference was seen between 22C3 and SP142 antibodies. PDL1+ cfRNA (N=59), PDL1− cfRNA (N=38). Positive concordance for cfRNA PDL1+ and IHC PDL1+ was 10.2% ( 6/59). Negative concordance for cfRNA PDL1− and IHC PDL1− was 97.4% (37/38) (p=0.079).

In ICIs ineligible patients a >50% IHC threshold is rarely met (4%). Random draw qPCR PDL1 is poorly correlated with IHC, with higher rates PDL1+ of cfRNA in setting of IHC PDL1−. In the other hand, cfRNA PDL1− is strongly associated with an IHC negative result. Further investigation incorporating both cfRNA and IHC result to predict, the response from are warranted.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise. Unless the context dictates the contrary, all ranges set forth herein should be interpreted as being inclusive of their endpoints, and open-ended ranges should be interpreted to include commercially practical values. Similarly, all lists of values should be considered as inclusive of intermediate values unless the context indicates the contrary.

Moreover, all methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

What is claimed is:

1. A method of treating a patient having cancer, comprising:
tailoring an immune therapy to treat the patient, and administering the tailored immune therapy to the patient,
wherein the tailored immune therapy is generated by,
obtaining whole transcriptomic sequencing data from a single tumor of the patient;
identifying, from the whole transcriptomic sequencing data, presence and/or activity of immune competent cells in the tumor;
identifying, from the whole transcriptomic sequencing data, expression level of immune checkpoint markers;
correlating (a) the presence and/or activity of immune competent cells, with (b) the expression level of immune checkpoint markers to tailor a tumor treatment for the patient.

2. The method of claim 1 wherein identifying expression level comprises determining over-expression or under-expression for the one or more immune regulatory proteins relative to respective reference ranges, wherein the reference ranges are specific for a specific tumor type.

3. The method of claim 2 wherein the immune checkpoint marker is annotated as expressed when the quantified expression level exceeds +/−2SD of the reference range.

4. The method of claim 2 wherein the reference ranges are specific for a specific tumor type as classified in ICD10.

5. The method of claim 1 wherein the immune checkpoint marker is selected from the list comprising of PDL1, PDL2, CTLA4, IDO1, LAG3, and TIM3.

6. The method of claim 1 further comprising a step of administering a treatment for the tumor.

7. The method of claim 6 wherein a PDL1 inhibitor therapy is administered for a PDL1-high tumor.

8. The method of claim 6 wherein a DO- or TIM3-directed therapy is administered for a PDL1-low tumor.

9. The method of claim 1 wherein the expression level of immune checkpoint markers is determined from a cfRNA sample obtained from blood of the patient.

10. The method of claim 1 wherein the tumor is breast cancer, colon cancer, lung cancer, pancreatic cancer, ovarian cancer, brain cancer, and/or prostate cancer.

11. A method of priming a patient for immune therapy of a tumor, comprising:
quantifying or obtaining expression levels for a plurality of somatic-specific single nucleotide variants (SNVs) from paired tumor and normal whole exome sequencing;
predicting MHC1 binding affinity for neoepitope peptides resulting from said SNVs, wherein neoepitope binding to MHC1 results in silenced neoepitopes;
identifying the patient for priming upon prediction of silenced neoepitopes; and
administering the identified patient with epigenetic priming therapy prior to immune therapy.

12. The method of claim 11 wherein the cancer is to breast cancer, colon cancer, lung cancer, pancreatic cancer, ovarian cancer, brain cancer, and/or prostate cancer.

13. The method of claim 11 wherein the SNV is annotated as expressed if observed in more than two RNAseq reads.

14. The method of claim 11 further comprising identifying a patient for the priming therapy, comprising:
quantifying or obtaining expression levels for a plurality of distinct genes, wherein the distinct genes are associated with respective distinct types of immune cells;
determining over-expression or under-expression for each of the distinct genes relative to respective reference ranges, wherein the reference ranges are specific for a specific tumor type; and
using the over-expression and/or under-expression of each of the distinct genes to infer activity and/or infiltration by the immune cells in the tumor, and
wherein patients with high immune infiltration are identified for the priming therapy.

15. The method of claim 11 wherein the patient identified for priming therapy has low PDL1 expression.

16. The method of claim 11 wherein the immune therapy comprises treatment with an immune checkpoint inhibitor.

17. The method of claim 11 wherein the immune therapy comprises treatment with at least one of a vaccine composition and an immune stimulatory cytokine.

18. A method of predicting an effective cancer therapy for a patient, comprising:
sequencing whole genomic and transcriptomic data to obtain in silico expressed neoantigens and expressed checkpoint markers;
predicting that a checkpoint therapy is an effective cancer therapy for the patient when the in silico sequencing data comprises (a) expressed neoantigens that are predicted to bind MEW, and (b) expressed checkpoint markers;
predicting that a combination of a DNA hypomethylating agent and checkpoint therapy is an effective cancer therapy for the patient when the in silico sequencing data comprises neoantigens that are predicted to bind MEW, but binding is suppressed by methylation; and
predicting that a combination of a HDAC inhibitor and checkpoint therapy is an effective cancer therapy for the patient when the in silico sequencing data comprises neoantigens that are predicted to bind MHC, but binding is suppressed by heterochromatin remodeling.

19. The method of claim 18 wherein the DNA hypomethylating agent is 5-aza-2′-deoxycytidine (5-AZA-CdR).

20. The method of claim 18 wherein the heterochromatin remodeling agent comprises Trichostatin A (TSA), trapoxin and/or depudesin.
